(19) United States
(12) Patent Application Publication
(10) Pub. No.: US 2008/0204660 A1
(43) Pub. Date: Aug. 28, 2008

(54) Ophthalmoscopic attachment module and surgical microscope having an ophtalmoscopic attachment module

(76) Inventor: Andreas Obrebski, Oberkochen (DE)

Correspondence Address: WALTER OTTESEN, PO BOX 4026, GAITHERSBURG, MD, 20885-4026, US

(21) Appl. No.: 11/889,686

(22) Filed: Aug. 15, 2007

(30) Foreign Application Priority Data
Aug. 16, 2006 (DE) 10 2006 038 911.5

(51) Int. Cl. A61B 3/13

(52) U.S. Cl. 351/221

(57) ABSTRACT

An opthalmoscopic attachment module (150) is provided for attachment to a surgical microscope (100). The opthalmoscopic attachment module (150) has a holding device (153) for an opthalmoscopic magnifier lens (154). This holding device (153) carries a device (180, 181) for guiding illuminating light (125). The unit (180, 181) guides the illuminating light past the opthalmoscopic magnifier lens (154) to an object region (190).

### CROSS REFERENCE TO RELATED APPLICATION

This application claims priority of German patent application no. 10 2006 038 911.5, filed Aug. 18, 2006, the entire content of which is incorporated herein by reference.

### FIELD OF THE INVENTION

The invention relates to an opthalmoscopic attachment module for attachment to a surgical microscope which has a holding device for an opthalmoscopic magnifier lens. The holding device carries an arrangement for guiding illuminating light.

### BACKGROUND OF THE INVENTION

A surgical microscope having an opthalmoscopic attachment module of the kind referred to above is disclosed in German utility model application 94 15 219 U1. In this publication, an attachment unit for a stereoscopic surgical microscope is described as the opthalmoscopic attachment module. This attachment unit functions for viewing the ocular fundus of the eye of a patient. The attachment unit includes an opthalmoscopic magnifier in the form of an attachment lens. The attachment lens is accommodated in a holding unit with the opthalmoscopic ancillary module directly above a patient eye to be examined. The opthalmoscopic magnifier lens generates an intermediate image of the ocular fundus on the side facing away from the object to be examined. This intermediate image can be viewed with the surgical microscope to which the opthalmoscopic attachment module is connected. The opthalmoscopic attachment module includes a joint for the holding device of the opthalmoscopic magnifier lens. The opthalmoscopic magnifier lens can be pivoted into and out of the viewing beam path of the corresponding surgical microscope on this joint.

It is possible to use the opthalmoscopic attachment module, which is described in German utility model application 94 15 219 U1, on surgical microscopes wherein an illuminating light for the region of the object is made available with this illuminating light passing through the microscope main objective. One such microscope is, for example, the OPMI® Visu 200 of Carl Zeiss AG. If, in this surgical microscope, the opthalmoscopic magnifier lens is pivoted into the stereoscopic viewing beam path forward of the microscope main objective, then the illuminating light passes through the opthalmoscopic magnifier lens into the patient eye and illuminates the ocular fundus.

If the illuminating beam path is guided through the opthalmoscopic magnifier lens, then disturbing light reflections can result in the viewed image of the surgical microscope which originate from the illuminating light which is reflected on the opthalmoscopic magnifier lens. Such light reflections are that much more pronounced with a contaminated opthalmoscopic magnifier lens, such as from droplets of blood. Contamination of the opthalmoscopic magnifier lens cannot, however, always be precluded in the course of a surgical procedure.

### SUMMARY OF THE INVENTION

In view of the above, it is an object of the invention to provide an opthalmoscopic attachment module which is suitable for working with a surgical microscope wherein the illuminating light is made available by passage through the microscope main objective and wherein nonetheless no disturbing reflections occur which could result from scattering of illuminating light on an opthalmoscopic magnifier lens.

The above object is achieved by the opthalmoscopic attachment module of the invention in that the arrangement for guiding the illuminating light guides illuminating light past the opthalmoscopic magnifier lens to the object region.

In this way, it is possible to match the illuminating beam path well to an object such as a patient's eye which is to be examined. Especially it is ensured that the position of the opthalmoscopic magnifier lens has no effect on the angle of incidence of the illuminating light which is guided into the object region to be examined.

According to a further feature of the invention, the arrangement for guiding the illuminating light has, at least in part, adjustable optical elements. In this way, the angle of incidence for the illuminating light is adjusted to a patient's eye for investigation by means of the opthalmoscopic attachment module. Accordingly, it is possible to adjust the course of the illuminating beam path which reaches the region of the object with this course being the most favorable for the examination of the object region.

In another embodiment of the invention, drives are assigned to the at least partially adjustable optical elements. In this way, it is possible to provide an automated adjustment of favorable illumination configurations in the opthalmoscopic attachment module so that this module can be easily and ergonomically favorably manipulated. Furthermore, drives make possible to provide a control loop for the at least partially adjustable optical elements and this control loop evaluates the image of a viewed region and optimizes the course of an illuminating beam path in the opthalmoscopic attachment module, if required, during continuous surgical procedure to provide an especially contrast rich and light intense image.

In another embodiment of the invention, the arrangement for guiding the illuminating light includes one or several mirrors, one or several light conductors, one or several lens elements or a deflection prism. In this way, it is possible to guide illuminating light to an object region, which is to be examined, without scattering losses.

Another embodiment of the invention provides for a light source integrated into the opthalmoscopic attachment module. In this way, the opthalmoscopic attachment module can be used in surgical microscopes wherein no illuminating unit is provided which outputs illuminating light.

In still another embodiment of the invention, an illuminating in-coupling unit is provided in the opthalmoscopic attachment module in order to take up illuminating light made available by the surgical microscope. Preferably, the illuminating light in-coupling unit is designed for taking up illuminating light which passes through the microscope main objective of the surgical microscope.

A surgical microscope having a corresponding opthalmoscopic attachment module makes possible a viewing of the ocular fundus of the eye of a patient without disturbing reflections.

### BRIEF DESCRIPTION OF THE DRAWINGS

The invention will now be described with reference to the drawings wherein:

FIG. 1 shows a surgical microscope having an opthalmoscopic attachment module wherein a deflection prism is integrated for guiding the illuminating beam path;

FIG. 2 is a section view of the opthalmoscopic attachment module shown in FIG. 1 viewed along the line II-II of FIG. 1;

FIG. 3 is a schematic of a surgical microscope having an opthalmoscopic attachment module wherein a light conductor is provided for guiding the illuminating beam path;

FIG. 4 shows a surgical microscope having an opthalmoscopic attachment module according to an alternate embodiment with the opthalmoscopic attachment module being provided with a light conductor in the illuminating beam path to guide the illuminating light to the object region;

FIG. 5 shows a surgical microscope having an opthalmoscopic attachment module which has a mirror system for guiding the illuminating light;

FIG. 6 is a schematic of a surgical microscope having an alternate embodiment for an opthalmoscopic attachment module wherein a mirror system is provided for guiding the illuminating light; and,

FIG. 7 is a schematic of a surgical microscope having an opthalmoscopic attachment module which has an integrated light source.

### DESCRIPTION OF THE PREFERRED EMBODIMENTS OF THE INVENTION

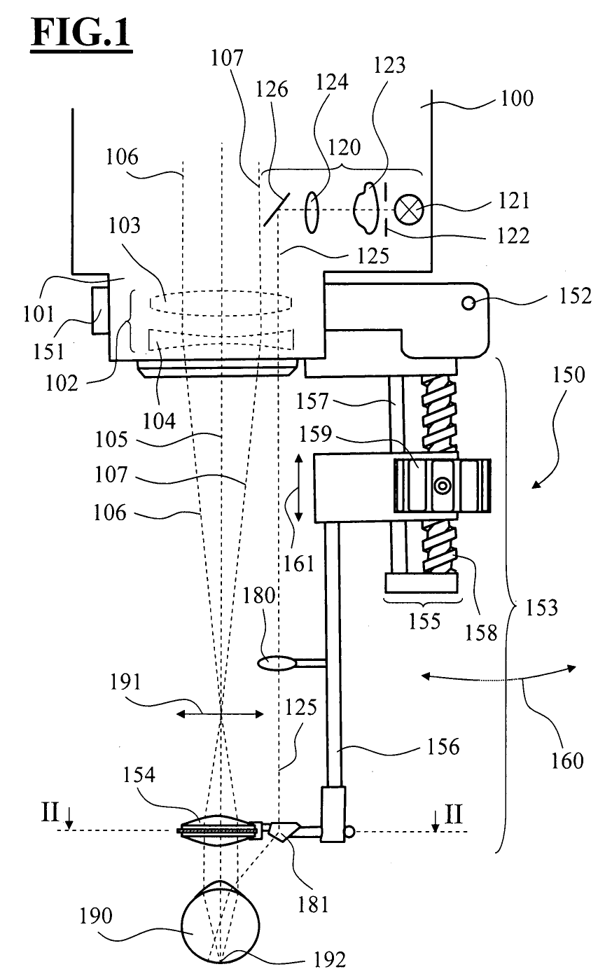

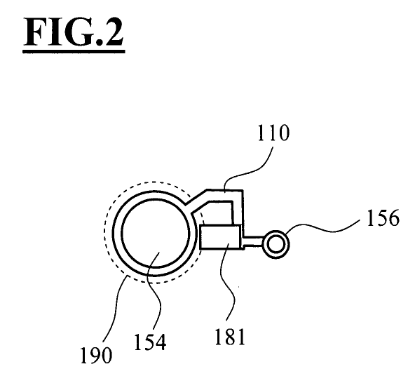

In FIG. 1, a surgical microscope 100 is shown on which an opthalmoscopic attachment module 150 is attached. The opthalmoscopic attachment module 150 is arranged with a holding device 151 on a receptacle 101 for a focusable microscope main objective system 102. The microscope main objective system 102 includes a lens 103 having a positive refractive power and a lens 104 having a negative refractive power. The lens 104 can be moved relative to the lens 103 in order to adjust the focus plane of the surgical microscope 100. The microscope main objective system 102 has an optical axis 105 and a stereoscopic viewing beam path (106, 107) passes through this main objective system. The viewing beam path (106, 107) is guided in the surgical microscope 100 through a magnification system (not shown) in order to make possible viewing the object region through an ocular (likewise not shown in FIG. 2).

The opthalmoscopic attachment module 150 has a joint 152. A holding device 153 with an opthalmoscopic magnifier lens 154 is accommodated in the joint 152. The holding device 153 with the opthalmoscopic magnifier lens 154 can be pivoted in the joint 152 corresponding to the double arrow 160 into and out of the viewing beam path (106, 107) of the surgical microscope 100.

The holding device 153 has a first section 155 and a second section 156. The first section 155 includes a guide rod 157 and a spindle drive 158. The second section 156 is held on the first section 155. The second section can be moved in correspondence to the double arrow 161 by moving the nut 159. The opthalmoscopic magnifier lens 154 is accommodated on the second section 156. By moving the nut 159, the distance of the opthalmoscopic magnifier lens 154 from the patient eye 190, which is to be examined, can be adjusted.

The opthalmoscopic magnifier lens 154 generates an intermediate image 191 of the ocular fundus 192 of the eye 190 of the patent. If the microscope main objective 102 is focused on the intermediate image 191, the ocular fundus 192 of the patient eye 190 can be sharply viewed in the surgical microscope 100.

The surgical microscope 100 has an illuminating system 120 having a light source 121. The light source makes available an illuminating light 125 via a field diaphragm 122 and lens elements 123 and 124 which reaches a path-folding element 126. The path-folding element 126 guides the illuminating light 125 through the microscope main objective system 102 to the eye 190 of the patient, that is, the object region.

A lens 180 having a positive refractive power is provided in the second section 156 of the holding device 153 for the illuminating light 125 which exits from the microscope main objective system 102. The lens 180 functions as an illuminating light in-coupling unit. The lens 180 collects the illuminating light 125, which passes through the microscope main objective system 102, and conducts this light to a deflection prism 181. The deflection prism 181 guides the illuminating light 125 laterally past the opthalmoscopic magnifier lens 154 and directs the illuminating light to the patient eye 190. The bypassing of the opthalmoscopic magnifier lens 154 by the illuminating beam path ensures that no disturbing reflections from the illuminating light can arise at the opthalmoscopic magnifier lens 154 which would then again be taken up in the viewing beam path (106, 107) of the surgical microscope.

FIG. 2 shows a section of the opthalmoscopic attachment module 150 along the line II-II in FIG. 1. The second section 156 of the opthalmoscopic attachment module carries the deflection prism 181 at its end, which faces toward the object region, and carries the opthalmoscopic magnifier lens 154 on a holding strut 110.

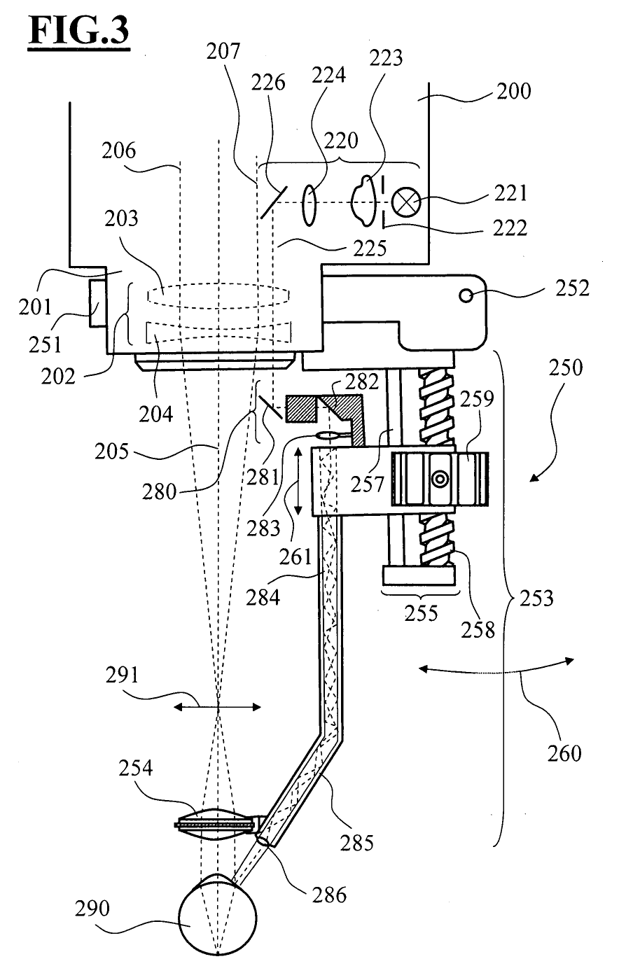

In FIG. 3, a surgical microscope 200 having an opthalmoscopic attachment module 250 is shown. The opthalmoscopic attachment module 250 has a holding device 251 which, in turn, is arranged on an annularly-shaped receptacle 201 for the microscope main objective system 202 of the surgical microscope. The microscope main objective system 202 is held so as to be focusable and has a lens 203 having a positive refractive power and a lens 204 having a negative refractive power. The opthalmoscopic attachment module 250 has a holding device 253 which is accommodated in a joint 252. The holding device 253 carries an opthalmoscopic magnifier lens 254. In the joint 252, the opthalmoscopic magnifier lens 254 can be pivoted on the holding device 253 into and out of the viewing beam path 205 of the surgical microscope 200 in correspondence to the double arrow 260.

The holding device 253 has, in turn, a first section 255 and a second section 285. The first section 255 includes a guide rod 257 and a spindle drive 258. The second section 285 is held on the first section 255. The second section 285 is configured as a light conductor 284 and can be moved by moving the nut 259 in correspondence to the double arrow 261.

In the surgical microscope 200, an illuminating system 220 is provided having a configuration corresponding to that of the illuminating system 120 in the surgical microscope 100 of FIG. 1. The component assemblies of the illuminating system, which are shown in FIG. 1, are identified by reference numerals in FIG. 2 which are increased by 100 in comparison to FIG. 1.

The surgical microscope 200 has an illuminating light in-coupling unit 280 which includes a path-folding mirror 281 and a path-folding mirror 282. If, as shown in FIG. 2, the holding device 253 is pivoted with the opthalmoscopic magnifier lens 254 into the viewing beam path (206, 207) of the surgical microscope 200, then the illuminating light 225 from the microscope main objective system is caught by path-folding mirror 281 and is coupled into a light conductor 284 via a path-folding mirror 282 and through a lens element 283. The light conductor 284 functions as a holding device and carries the opthalmoscopic magnifier lens 254. The light conductor 284 is accommodated in a section 255 of the holding device 253 and can be displaced by moving a nut 259 in such a manner that the opthalmoscopic magnifier lens 254 can be raised and lowered in correspondence to the double arrow 261. In a section 285 directed toward the object region, the light conductor 284 is configured so as to be bent. The light conductor 284 conducts the illuminating light conducted therethrough through a lens element 286 to reach the object to be examined which is in the form of the eye 290 of a patient.

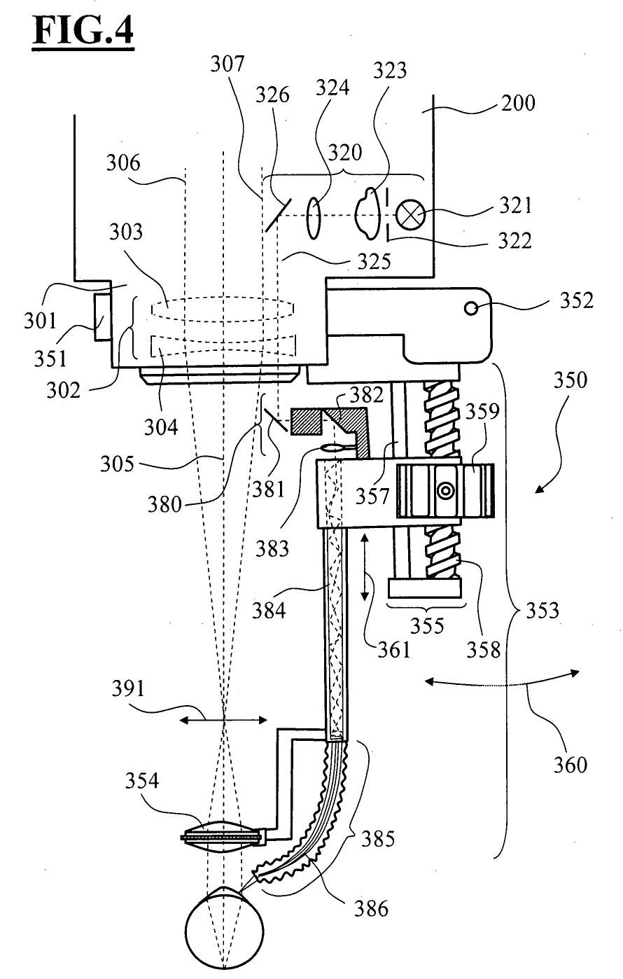

FIG. 4 shows a surgical microscope 300 having an opthalmoscopic attachment module 350 having a configuration corresponding essentially to the surgical microscope 200 with the opthalmoscopic attachment module 250 of FIG. 3. The assembly groups, which are identical in FIGS. 3 and 4 for the surgical microscope having the opthalmoscopic attachment module, have reference numerals in FIG. 4 increased by 100 in comparison to FIG. 3. In the opthalmoscopic attachment module 350, a light conductor 384 is provided which has a section 385 configured to have a swan-neck shape. This swan-neck shaped section 385 contains a fiber optic 386. The section 385 is movable in the same manner as a garden hose and its course can be adjusted. The section 385 retains that form which is once adjusted by an operator.

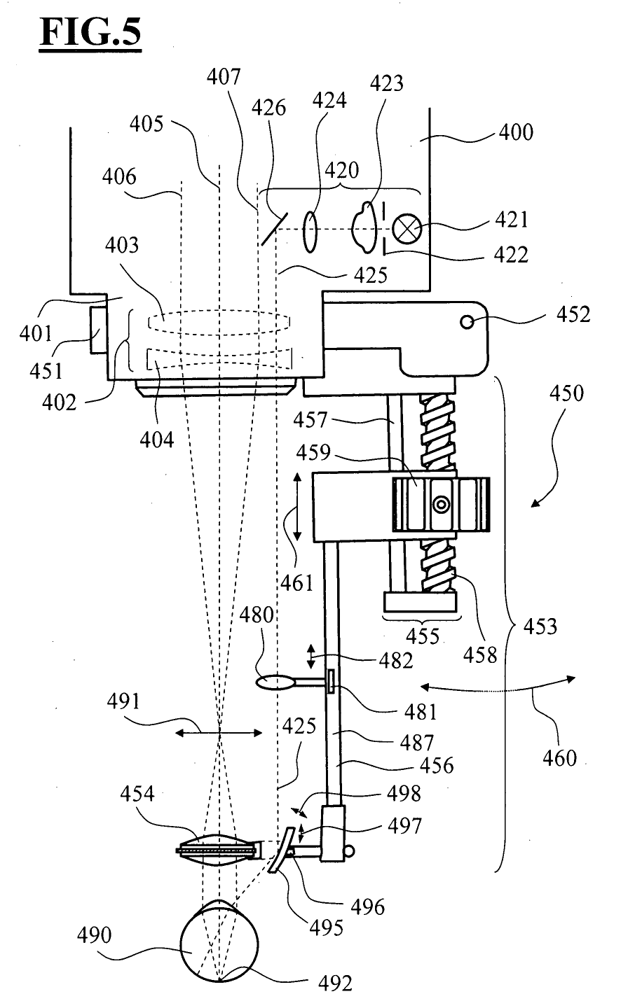

FIG. 5 shows a surgical microscope 400 having an opthalmoscopic attachment module 450. The configuration of the surgical microscope 400 having the opthalmoscopic attachment module 450 corresponds to that of surgical microscope 100 and opthalmoscopic attachment module 150 of FIG. 1. Component assemblies and items which correspond to each other are therefore increased by the number 300 in FIG. 5 compared to FIG. 1.

What is different in the opthalmoscopic attachment module 150 from FIG. 1 is that the opthalmoscopic attachment module 450 has no deflection prism; instead, a mirror element 495 is provided. A piezo drive 496 is assigned to this mirror element 495. The piezo drive makes possible that the mirror element 495 can be shifted in correspondence to the double arrows 497 and 498. At the same time, a drive 481 is assigned to the lens element 480. This drive makes it possible to move the lens element 480 along the section 487 of the holding device 453 in correspondence to the double arrow 482.

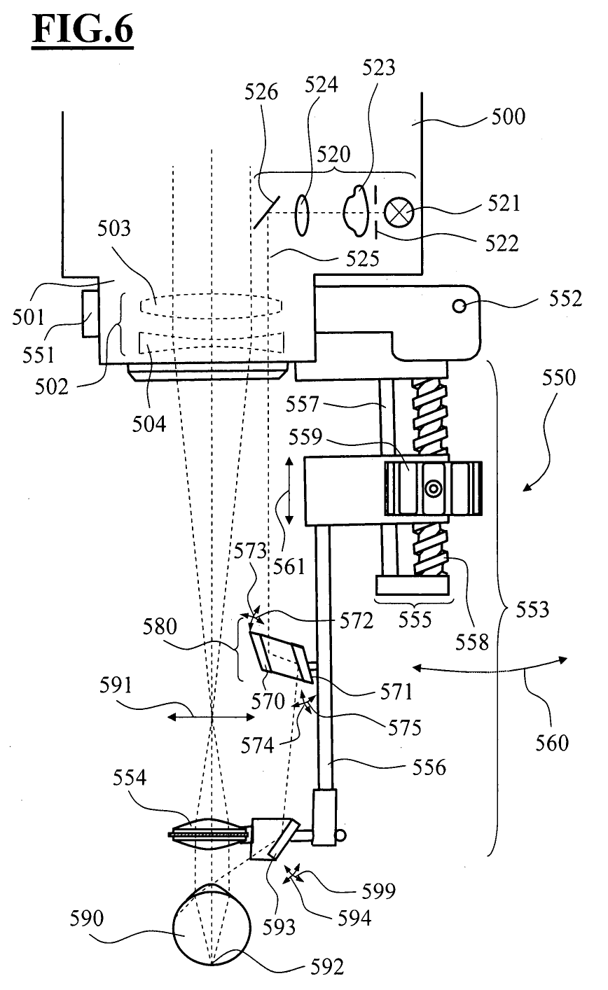

FIG. 6 shows a surgical microscope 500 having an opthalmoscopic attachment module 550 which has a mirror system in order to direct illuminating light 525 into a patient eye 590 to be examined. The illuminating light 525 is made available by means of an illuminating system 520 in the surgical microscope 500. Insofar as the component assemblies and items in FIGS. 5 and 6 are identical, they have reference numerals in FIG. 6 increased by 100 with respect to FIG. 5.

A first curved mirror 570 and a second curved mirror 571 are provided in the opthalmoscopic attachment module 550. The mirrors 570 and 571 operate as illuminating light in-coupling unit 580 and catch the illuminating light 525 which passes through the microscope main objective system 502 of the surgical microscope 500. The illuminating light in-coupling unit 580 is configured to be adjustable in correspondence to the double arrows (572, 573, 574, 575) and is held on a second section 556 of the holding device 553. The illuminating light in-coupling unit 580 directs illuminating light 525 to the path-folding mirror 593 which can be shifted in correspondence to the double arrows (594, 599). This path-folding mirror 593 guides the illuminating light past the opthalmoscopic magnifier lens 554 to the patient eye 590.

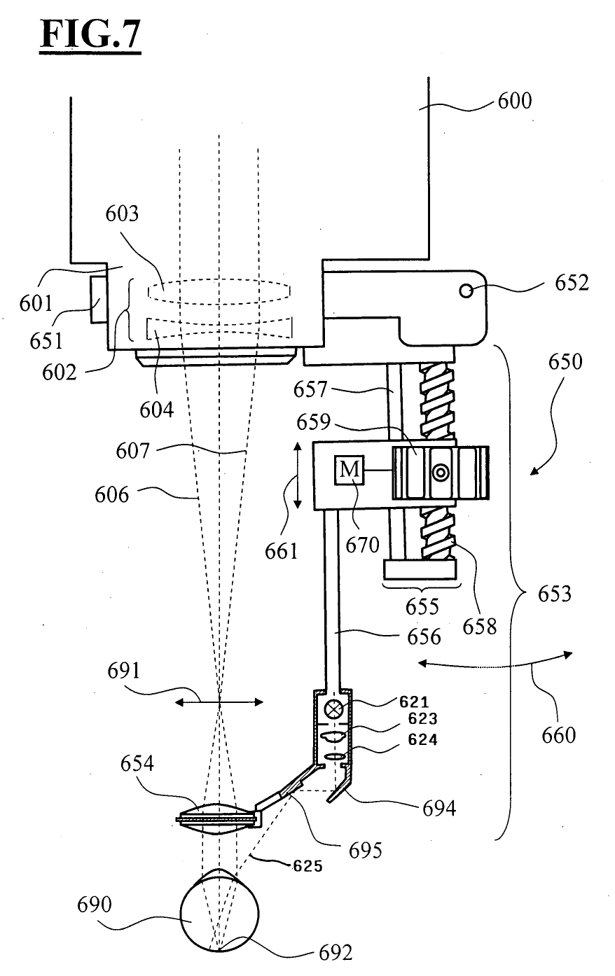

In FIG. 7, a surgical microscope 600 having opthalmoscopic attachment module 650 is shown wherein, as with the opthalmoscopic attachment module 150 of FIG. 1, the opthalmoscopic attachment module 650 is mounted by means of a holding device 651 on a receptacle 601 for a focusable microscope main objective system 602. The microscope main objective system 602 has a lens 603 having positive refractive power and a lens 604 having negative refractive power. A joint 652 is provided in the opthalmoscopic attachment module 650. In the joint 652, a holding device 653 is held which carries an opthalmoscopic magnifier lens 654. The opthalmoscopic magnifier lens 654 can be pivoted into and out of the viewing beam path (606, 607) of the surgical microscope 600 in correspondence to the double arrow 660 in the same manner as the opthalmoscopic magnifier lens 154 in the opthalmoscopic attachment module 150 of FIG. 1.

The holding device 653 has a first section 655 with a guide rod 657 and with a threaded spindle 658 on which a nut 659 acts. The first section 655 carries a second section 656 on which the opthalmoscopic magnifier lens 654 is accommodated. An adjusting motor 670 is assigned to the nut 659. By means of the adjusting motor 670, the nut 659 can be controllably driven whereby the second section 656 of the holding device 653 can be moved relative to the first section 655 in correspondence to the double arrow 661. A light source 621 having an illuminating optic is integrated into the second section 656. This light source includes lens elements 623 and 624. The light source 621 is supplied with electric energy via feed lines (not shown) from the surgical microscope 600. Alternatively, it is also possible to provide a battery feed for the light source.

The illuminating light 625, which is provided by light source 621, is, after it is guided through the illuminating optic, conducted to mirror surfaces 694 and 695. These mirror surfaces (694, 695) direct the illuminating light 625 past the opthalmoscopic magnifier lens 654 to the object region in the form of the patient eye 690.

It is understood that the foregoing description is that of the preferred embodiments of the invention and that various changes and modifications may be made thereto without departing from the spirit and scope of the invention as defined in the appended claims.

What is claimed is:

1. An opthalmoscopic attachment module for attaching to a surgical microscope for viewing an object region, the opthalmoscopic attachment module comprising:
a holding device;
an opthalmoscopic magnifier lens mounted on said holding device;
an arrangement for guiding an illuminating light; and,
said arrangement being carried by said holding device and being configured to guide said illuminating light past said opthalmoscopic magnifier lens to the object region.

2. The opthalmoscopic attachment module of claim 1, said arrangement including at least partially adjustable optical elements for guiding said illuminating light.

3. The opthalmoscopic attachment module of claim 2, said arrangement further including drives assigned to said optical elements for adjusting said optical elements.

4. The opthalmoscopic attachment module of claim 1, said arrangement including one or a plurality of mirrors for guiding said illuminating light.

5. The opthalmoscopic attachment module of claim 1, said arrangement including one or a plurality of light conductors for guiding said illuminating light.

6. The opthalmoscopic attachment module of claim 1, said arrangement including one or a plurality of lens elements for conducting said illuminating light.

7. The opthalmoscopic attachment module of claim 1, said arrangement including a deflection prism for deflecting said illuminating light.

8. The opthalmoscopic attachment module of claim 1, further comprising a light source integrated therein.

9. The opthalmoscopic attachment module of claim 1, further comprising an illuminating light in-coupling unit for gathering illuminating light made available by said surgical microscope.

10. The opthalmoscopic attachment module of claim 9, wherein said surgical microscope has a main objective through which said illuminating light passes; and, said illuminating light in-coupling unit is adapted to gather said illuminating light passing through said main objective.

11. A surgical microscope assembly comprising:
a surgical microscope for viewing an object region;
an opthalmoscopic attachment module mounted on said surgical microscope; and,
said opthalmoscopic attachment module including: a holding device; an opthalmoscopic magnifier lens mounted on said holding device; and, an arrangement for guiding an illuminating light; and,
said arrangement being carried by said holding device and being configured to guide said illuminating light past said opthalmoscopic magnifier lens to the object region.

12. The surgical microscope assembly of claim 11, said arrangement including at least partially adjustable optical elements for guiding said illuminating light.

13. The surgical microscope assembly of claim 12, said arrangement further including drives assigned to said optical elements for adjusting said optical elements.

14. The surgical microscope assembly of claim 11, said arrangement including one or a plurality of mirrors for guiding said illuminating light.

15. The surgical microscope assembly of claim 11, said arrangement including one or a plurality of light conductors for guiding said illuminating light.

16. The surgical microscope assembly of claim 11, said arrangement including one or a plurality of lens elements for conducting said illuminating light.

17. The surgical microscope assembly of claim 11, said arrangement including a deflection prism for deflecting said illuminating light.

18. The surgical microscope assembly of claim 11, further comprising a light source integrated into said opthalmoscopic attachment module.

19. The surgical microscope assembly of claim 11, further comprising an illuminating light in-coupling unit for gathering illuminating light made available by said surgical microscope.

20. The surgical microscope assembly of claim 19, wherein said surgical microscope has a main objective through which said illuminating light passes; and, said illuminating light in-coupling unit is adapted to gather said illuminating light passing through said main objective.
